(19) United States
(12) Patent Application Publication
(10) Pub. No.: US 2003/0077300 A1
(43) Pub. Date: Apr. 24, 2003

(54) System and method for intranasal administration of opioids

(76) Inventor: Daniel P. Wermeling, Lexington, KY (US)

Correspondence Address: Kalow & Springut LLP, 19th Floor, 488 Madison Avenue, New York, NY, 10022, US

(21) Appl. No.: 10/155,624

(22) Filed: May 24, 2002

Related U.S. Application Data
(63) Continuation of application No. 09/569,125, filed on May 10, 2000.

(51) Int. Cl. A61K 31/485; A61L 9/04; A61K 9/00

(52) U.S. Cl. 424/400; 424/45; 514/282

(57) ABSTRACT

The invention relates to pharmaceutical drug compositions and preparations that are narcotic antagonists and analgesics, specifically opioids, more specifically morphine and its pharmaceutically active derivatives, analogues, homologues, and metabolites, and still more specifically hydromorphone and butorphanol. This invention also relates to pharmaceutical drug delivery devices, specifically to devices for the intranasal administration of drugs classified as controlled substances. The invention also relates to the field of acute pain management through pharmaceutical intervention, particularly as practiced in an institutional setting, such as a hospital.

### FIELD OF THE INVENTION

The invention relates to pharmaceutical drug compositions and preparations that are narcotic antagonists and narcotic analgesics, specifically opioids, more specifically morphine and its pharmaceutically active derivatives, analogues, homologues, and metabolites, and still more specifically hydromorphone and butorphanol. This invention also relates to pharmaceutical drug delivery devices, specifically to devices for the intranasal administration of drugs classified as controlled substances. The invention also relates to the field of acute pain management through pharmaceutical intervention, particularly as practiced in an institutional setting, such as a hospital.

### BACKGROUND OF THE INVENTION

Marketers of opioids and other therapeutic compounds that act as systemic analgesics that have been approved by the U.S. Food and Drug Administration (“FDA”) and long used for oral, intramuscular and/or intravenous administration, have generally not sought regulatory approval from the FDA for liquid compositions of the same therapeutic compound for intranasal administration. This is surprising since it is well-known from the literature that the intranasal administration of a pharmacologically active compound generally results in a more rapid bioavailability of the compound, or of its desired active metabolite than if the compound is administered orally. Moreover, the total quantitative dosage required to achieve the same concentration of the active compound in the bloodstream is generally less via the intranasal route compared to oral administration, because in oral administration a portion of the active compound is often converted to a non-active metabolite by passage through the GI tract and in the liver.

The intranasal route of administration also provides numerous advantages over intravenous (IV) and intramuscular (IM) injections. One principal advantage of intranasal administration is convenience. An injectable system requires sterilization of the hypodermic syringe and in the institutional setting, leads to concerns among medical personnel about the risk of contracting disease if the they are accidentally stuck by a contaminated needle. Strict requirements for the safe disposal of the used needle and syringe must also be imposed in the institutional setting In contrast, intranasal administration requires little time on the part of the patient and the attending medical personnel, and is far less burdensome on the institution than injectables. There is no significant risk of infection of medical personnel or others in the institutional setting that is associated with nasal spray devices.

A second important advantage of intranasal administration over IM and IV is patient acceptance of the drug delivery system. Many, if not most, patients experience anxiety and exhibit symptoms of stress when faced with hypodermic injections via the IM or IV routes. In some cases, the after-effects of the injection include burning, edema, swelling, turgidity, hardness and soreness. In contrast, intranasal administration is perceived as non-invasive, is not accompanied by pain, has no after-effects and produces the gratification of prompt relief in the patient exhibiting the symptom. This is of particular advantage when the patient is a child. Most people have some familiarity with nasal sprays in the form of over-the-counter decongestants for alleviating the symptoms of colds and allergies, that they or a family member have used routinely. Another important consideration is that the patient can self-administer the prescribed dosage(s) of nasal spray. An empty nasal spray device, or one containing only saline solution or the like, can be given to the patient to practice the technique for proper insertion and activation for self-administration.

In view of the aforementioned advantages and benefits afforded by the intranasal administration, it would be expected that many known compounds exhibiting systemic pharmacological activity, including opioid analgesics, that have been approved for and commercially used for many years, would presently be available for intranasal administration. The only opioid available in an FDA approved intranasal manual-metering spray device is butorphanol sold by Bristol-Myers Squibb under the brand name STADOL®NS.

Butorphanol nasal spray dosage received FDA approval subsequent to the issuance of U.S. Pat. No. 4,464,378 which issued to Hussain in 1984 and is assigned to the University of Kentucky. The Hussain patent discloses various forms for nasal administration of this class of compounds, including ointments and gels, and suggests that liquid nasal solutions for use ad drops or sprays be formulated. However, Hussain disclosed in vitro test results only on rats and no human test data or results are provided. In a comparative study three groups of three rats each were administered the drug naloxone by IV injection, orally (via injection directly through the duodenum) and nasally by injecting a liquid solution from a syringe via a polyethylene tube surgically inserted into the rat's nasal cavity. Blood samples were drawn from the femoral cavity to determine plasma levels of the drug.

Despite the remarkable commercial success that has been enjoyed by those drugs that have been made available in intranasal form, in fact, only a very limited number of compounds are commercially available to physicians to prescribe and dispense to their patients in that form. No opioids or other controlled substances have heretofore been made available as intranasal formulations.

Only one multiple-dose spray device has apparently been approved by the FDA for intranasal administration of an opioid solution that is categorized as controlled substance. The devices that are presently available exhibit several deficiencies. One spray device intended for multiple uses must be primed before use by expelling a portion of the liquid contents in order to assure that the pump mechanism and delivery tube are filled. Up to seven or eight activations are required to prime the device. It is also indicated that further priming to disperse one or two sprays is to be performed if the device is not used for 48 hours or longer. These procedures necessarily result in the dispenser being overfilled in order to assure that there will be sufficient liquid to deliver the labelled number of doses. It has been found that a substantial volume of the controlled substance often remains in the device, even after the labelled number of doses have been administered. In practice, it has also been found that medical personnel and workers at health care facilities routinely abscond with the dispensers, sometimes after the patient has had only one or a few of the prescribed doses in a multi-dose container. This improper diversion and use of controlled substances as so-called “recreational drugs” is well-known among medical facility managers and law enforcement authorities. So far as is presently known, no preventative measures have been reported that are effective in dealing with this problem.

A further problem resides in dispensing to a patient intranasal spray devices with sufficient fluid contents for numerous doses for pain control purposes. Because many analgesics based on opioids and other compounds produce a euphoric effect along with the relief of pain, the patient uses the medication more frequently than prescribed, providing the potential for overdosing. Moreover, because of the nature and construction of the multiple dose spray device, medical personnel cannot easily determine the number of doses that have been administered by a simple visual inspection of the device.

Another problem that has recently been identified in clinical studies is the relative inaccuracy of multi-dose intranasal delivery devices that are currently being marketed with opioid solutions for the control of pain. Not only does the average volume of liquid spray actually administered fall about 10% below the purported dosage appearing on the approved label for one such product, significant variations were also observed among a series of administrations by each patient in the study group. Thus, spray devices tested containing an opioid compound classed as a “controlled substance” by the FDA were found to be capable of administering only about 90% by volume of the prescribed dosage, on average, and the dosage actually received by each patient in repeated administrations exhibited substantial variations of from 60% to 130% of the claimed label dosage.

### OBJECTS OF THE INVENTION

Accordingly, it is a principal object of the invention to provide a novel therapeutic composition of an opioid or other synthetic or semi-synthetic systemic analgesic for intranasal administration of at least one predetermined volumetric unit dose by means that delivers the therapeutically prescribed unit dose or number of unit doses that are highly accurate as to the volume discharged and which leave no significant quantity of the composition in the delivery means.

Another object of the invention is to provide a novel composition comprising a known analgesic compound that is approved for oral, IM and/or IV administration for use in a highly accurate and reproducible intranasal delivery system in a single unit-dose or therapeutically prescribed multiple unit-dose.

It is another object of this invention to provide an intranasal delivery system for one or more unit doses of novel therapeutic analgesic compositions containing compounds classed as “controlled substances” that permits administration of one or more therapeutically prescribed unit-doses in a medical care facility, such as a hospital or day clinic, in which the delivery system contains essentially no significant quantity of the therapeutic composition after administration of the single unit-dose or the prescribed number of multiple unit-doses.

It is also an object of the invention to provide the novel and improved combination of a device of intranasal administration and a formulation for a systemic opioid analgesic that meet the requirements for FDA approval.

It is a further object of this invention to provide a dosage form and method of administration of an analgesic that exhibits a rapid onset, moderate duration of therapeutic activity, minimal side effects, predictable bioavailability, ease and safety of administration, and minimal physical discomfort and anxiety to the patient occasioned by administration.

Yet another object of the invention is to provide such novel compositions for intranasal administration in a relatively small and inexpensive, manually operated, self-contained hand-held disposable device that retains essentially no significant quantity of the therapeutic composition after administration of the one or more unit-doses as prescribed.

A further object of the invention is to provide a comprehensive method for providing a novel therapeutic composition for intranasal administration that contains one or more known pharmacologically active compounds that are approved for oral, IM and/or IV administration, the intranasal composition being available for delivery in highly accurate and reproducible predetermined unit-doses leaving essentially no significant quantity of the therapeutic composition after administration of the prescribed number of unit-doses.

As used herein, the term “essentially no significant quantity of the therapeutic composition” means none, or a trace amount, or an amount that is so small that it cannot be recovered for a subsequent unintended use or abuse after the prescribed use.

### SUMMARY OF THE INVENTION

The invention comprehends the intranasal administration of specific classes of pharmacologically active compositions in the form of a liquid for nasal instillation in a unit-dose of a predetermined therapeutic volume, where substantially all of the predetermined volume of the composition is delivered within a specified narrow range of accuracy, while leaving essentially no significant quantity of the therapeutic composition in the applicator from the unit-dose as administered. The dose is administered in the form of liquid droplets, an atomized mist or an aerosol, or in a form that is a combination of the above. The dose can also comprise microcrystalline particles of the pharmaceutically active composition in a form that is readily absorbable by the nasal mucosa and with no or minimal undesirable side effects.

The compositions administered in accordance with the method and system of the invention are most advantageously those which exhibit systemic pharmacological effects following absorption from the nasal mucosa.

The classes of compounds comprising the invention are those pharmaceutically active compounds that have been or will be approved by the FDA and are administered orally and/or by injection, including IM and IV, for the treatment of specified diseases, disorders and conditions, but which compounds have not been offered in such an accurate and controlled unit-dose delivery system for intranasal administration as described herein. Compounds that are readily absorbable by the nasal mucosa without damaging or irritating the mucosa, or producing an allergic, or other unacceptable reaction in the recipient are deemed to have utility in the practice of the invention.

The specific compounds intended for use in the compositions and the method and the delivery system in the practice of the invention include the following compounds: morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl.

Compounds for use in the practice of the invention must be soluble in a pharmacologically acceptable carrier that can be nasally administered with safety over the entire reasonably foreseeable range of prescribed users of the composition. The composition containing the active compound or compounds preferably has a shelf life in the chosen delivery system of at least six months, and most preferably greater than six months and are compatible with the delivery system. The composition for use in the invention are formulated to deliver the dose within the foreseeable temperature ranges of exposure, e.g., to without becoming too viscous to be administered in the proper form by the device; or crystallizing at lower temperatures, and without exceeding the internal pressure limits of the delivery system at higher temperatures.

Other criteria to be applied in the selection of active compounds for intranasal administration relate to the nature of the disease or condition and/or the symptom(s) to be treated, the expected frequency with which the patient must receive the treatment, the foreseeability or unpredictability of the need for treatment, the age and capability of the patient to self-administer the treatment, the overall number of prospective users of the treatment in the general population, evidence that other available forms of the pharmacological compound are being abused.

The predetermined therapeutic volume of the pharmaceutical composition contained in the unit dose is delimited by several parameters, including the capability of the nasal passage to receive and absorb the volumetric quantity of liquid; the solubility of the particular pharmaceutical compound in the physiologically and pharmacologically acceptable nasal carrier liquid at the concentration required to achieve the desired effect; and in the case of a crystalline compound and/or composition, the availability of a compatible and efficacious propellant and delivery system. The relative safety of administering a given predetermined quantity of the pharmaceutical composition to classes of patients whose body weight, age, general health, use of other medications and may vary widely and can be determined by methods well known in the art.

Dispensing devices meeting the above criteria and technical specifications are commercially available from several sources. Devices suitable for use in the practice of the invention are commercially available from Pfeiffer of America of Princeton, N.J. and Valois of America, Inc. of Greenwich, Conn. Such devices have the capability of consistently delivering a predetermined volumetric amount of a liquid composition intranasally via a unit-dose dispenser that is manually operable by the patient requiring such intranasal drug administration. These manually operable devices are designed for delivery of a single unit-dose, after which there is essentially no significant quantity of the therapeutic composition remaining in the device. The device can thereafter be discarded without concern that others may abuse the opioid or other controlled substance.

Commercial devices are provided with enough pharmacologically active composition to administer one predetermined unit-dose or two unit-doses (“bi-dose”), each with a high degree of accuracy and reproducibility for the device and among a plurality of such commercially manufactured and filled devices.

The currently available commercial devices that are suited for used in the practice of the invention are fabricated from a variety of polymeric materials, can include glass or polymer containers for the therapeutic liquid composition, and metal components that form elements of the delivery system. Such devices are compact, relatively inexpensive and can be discarded after the prescribed use.

In a preferred embodiment, the container and its sealing means are sterilizable; most preferably, the entire device is constructed and assembled in a configuration that can be sterilized. Devices with one or more unit-dose(s) can be sterilized either before or after packaging, employing methods and technology that are well known in the art. Individual devices can be packaged, sterilized and shipped; alternatively, entire shipping and storage packages can be sterilized at once, and the devices removed individually for dispensing without affecting the sterility of the remaining units.

### BRIEF DESCRIPTION OF THE DRAWINGS

The novel features and other advantages of the present invention, in addition to those mentioned above, will become apparent to those skilled in the art from the following detailed description and in conjunction with the accompanying drawings, in which:

FIG. 1 is a graphic representation of the concentration of butorphanol in blood plasma versus time;

FIG. 2 is a graphic representation of the data of FIG. 1 over a longer time period;

FIG. 3 is a graphic representation of the concentration of hydromorphone in blood plasma versus time for IV, IM and IN doses;

FIG. 4 is a graphic representation of the data of FIG. 3 over a longer period of time; and

FIG. 5 is a graphic representation of the concentration of hydromorphone in blood plasma versus time for a group of subjects.

### DETAILED DESCRIPTION OF THE PREFERRED EMBODIMENTS

The following study was undertaken in order to determine the relative accuracy by which an analgesic composition in accordance with the present invention is administered. This study included comparison with a prior art delivery system that is sold commercially for the intranasal administration of butorphanol for institutional use. The prior art delivery system is a multi-dose sprayer that purports by its label to administer a specified 0.1 gm of liquid composition by metering upon activation by the user. The prior art composition is sold commercially by Bristol-Myers Squibb under the trademark STADOL®NS.

The delivery system employed in accordance with the present invention was a unit-dose disposable intranasal applicator that is commercially available from Pfeiffer of America under the designation “Unitdose Second Generation.” Each of the Pfeiffer spray applicators was charged with sufficient liquid to deliver a 0.1. mL dose of the same STADOL® NS liquid composition and that was purchased from Bristol-Myers Squibb. The glass containers were filled using a pipette under clean conditions, sealed and assembled to the applicator.

Each of the applicators was weighed prior to use and after use. Qualified medical personnel took the respective applicators to patients in a clinical setting for whom the drug had been prescribed and attended each of the patient's self-administration, one dose up each nostril, after which the applicator was recovered for weighing. In the case of the unit-dose applicators (Pfeiffer), each patient used two devices, both of which were discarded following the post-use weighing. The results of these studies of the method and system of the invention and the comparative prior art method follow.

TABLE 1
Sample Characteristics of Dose Weight Delivery

| Delivery | | mean wt. | std. | std. | | |
|---|---|---|---|---|---|---|
| System | n | gms | dev. | error | minimum | maximum |
| Unit-Dose | 23 | 0.206 | 0.00660 | 0.00138 | 0.193 | 0.223 |
| Multi-Dose | 24 | 0.180 | 0.0285 | 0.00582 | 0.114 | 0.220 |

Unit-Dose:

The statistical comparison of dose 1 and dose 2 for the Pfeiffer unit dose delivery system was done using a paired t-test. Analysis of the data indicated that the difference between the mean sprays of the two applications using the Pfeiffer device was not statistically significant (t=1.0; p=0.3).

The sample of 23 sprayers (actually 23 sets of 2 sprayers, since they were single-dose) had a mean total dose for two sprays of 0.206 grams with a standard deviation of 0.00660 grams.

Multiple Dose:

The total dose dispensed by two sprays was recorded. The sample of 24 multi-dose sprayers had a mean total dose for two sprays of 0.180 grams with a standard deviation of 0.0285 grams.

Comparison of Average Total Dose:

The two-sample t-test for the comparison of the unit-dose and multi-dose sprayers indicated a statistically significant difference between the mean total doses taking into account the size of the sample. The unit-dose mean total dose was significantly closer to the prescribed target and dose than the multi-dose mean total dose (t=4.3; p<0.001). A 95% confidence interval for the difference in means is (0.0140, 0.0380).

Comparison of Variability:

The F test for the comparison of variances revealed that the variability in the total doses dispensed by the multi-dose sprayer was significantly higher than the variability in weights dispensed by the unit-dose sprayer (F=18.7; p<0.001). The variability in the multi-dose sprayer is 18.6 times that of the unit-dose sprayer.

High variability in dose delivery leads to higher rates of adverse drug effects at excessive dose and inadequate treatment if the dose is low. Both consequences harm the patient, hence the goal is to precisely deliver the prescribed dose.

Comparison of each Sprayer to the Standard of 0.2 Grams

A t-test was used in each case to compare the observed sample mean to the desired weight of 0.2 grams. The unit-dose sprayer dispensed a mean total weight that was significantly higher than the goal of 0.2 grams (t=4.4; p<0.001). A 95% confidence interval for the mean total weight dispensed by the unit-dose sprayer is (0.203, 0.209). The multi-dose sprayer dispensed a mean total weight that was significantly lower than the goal of 0.2 grams (t=3.4; p<0.003). A 95% confidence interval for the mean total weight dispensed by the multi-dose sprayer is (0.168, 0.192). Based on the above, the unit-dose delivery system in accordance with the invention exhibits a much higher degree of accuracy in intranasally administering the volume of liquid composition corresponding to 0.1 gm:+3% vs −10%.

Two further statistical analyses were undertaken based on data obtained from the above study. The first assesses the bioequivalence of butorphanol administered using two different delivery systems. The Pfeiffer device was considered the “test” formulation and Stadol® the “reference” formulation. The second analysis was to determine whether the intrasubject variabilities of the two formulations are equal. The study was initiated with 16 subjects, 15 of which completed the study to provide data for this analysis; one subject dropped out after the second period. The following analysis considers both raw and normalized data, with the latter standardized with respect to the dose dispensed. For both the raw and normalized data, log transformations are applied to the pharmacokinetic endpoints Cmax, AUC(last), and AUC(inf).

Bioequivalence

A mixed effects model was considered for each parameter. Fixed effects for the factors sequence (4 levels), period (3 levels) and formulation (2 levels) were included in the model. Additionally, gender, as well as the interactions between gender and each of sequence, period and formulation was included as a factor in each model to determine whether separate analyses would be necessary for males and females. A total of seven models were considered: Tmax, log of raw Cmax values, log of normalized Cmax values, log transformed values for raw and normalized AUC(last), and log values for raw and normalized AUC(inf). In all cases, the interaction between gender and formulation was not significant, indicating that separate models for males and females were not warranted. In addition, the lack of significance of the effects included in each model indicate that there was no evidence of unequal carryover between the delivery system of the prior art and that of the invention.

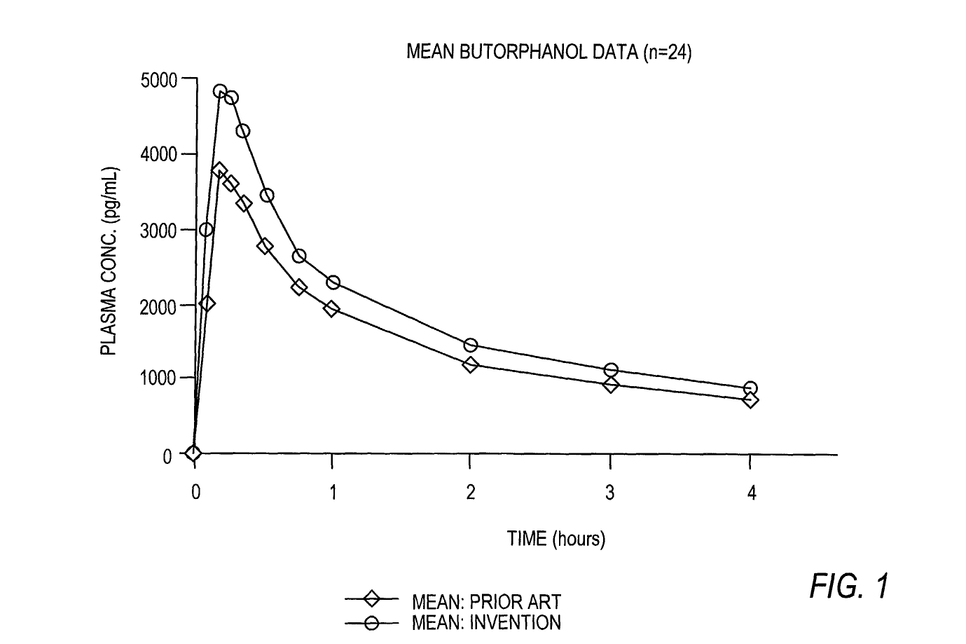

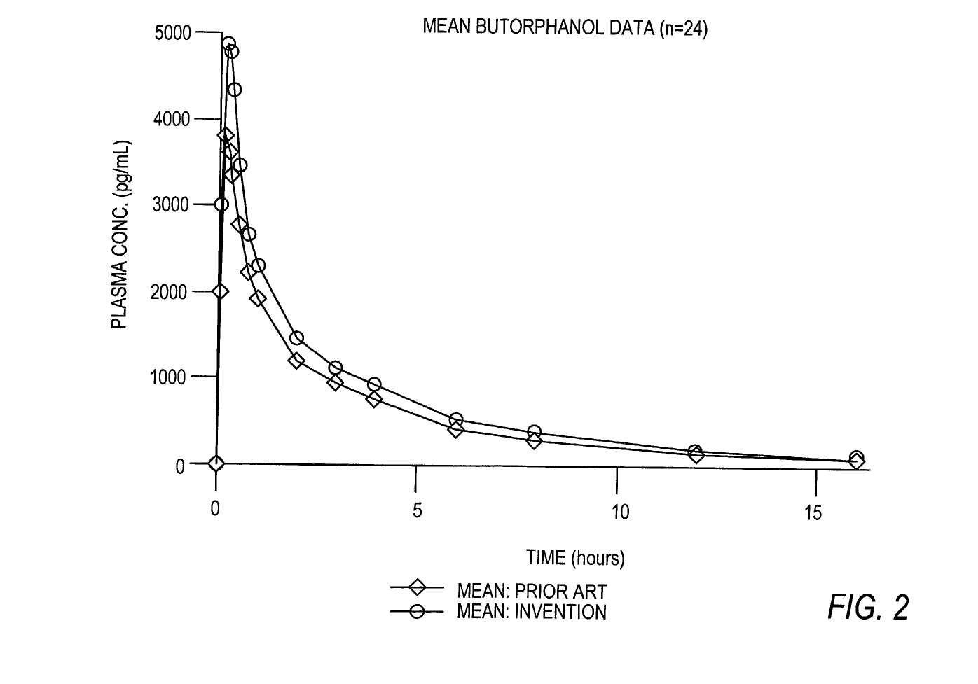

The mean levels of butorphanol from analysis of the subject's blood plasma reported in pg/ml is plotted against time in FIGS. 1 and 2. As would be expected from the data evidencing a much lower than label dosage for the prior art device, the concentration of the drug was significantly for the prior art method as compared to that of the invention.

The testing for bioequivalence was done using the method of two one-sided t-test (as described by Bolton, S., Pharmaceutical Statistics. Marcel Dekker, inc., New York, 1997, pages 415 ff.) For each parameter, the 90% confidence interval for the ratio of the test unit-dose to reference multi-dose formulations appear in Table 2 below.

TABLE 2
Summary of the two one-sided hypothesis tests for PK parameters

| | Lower Conf Limit for | Upper Conf Limit for |
|---|---|---|
| Parameter | Ratio of Test/Reference | Ratio of Test/Reference |
| Tmax | 0.749 | 1.132 |
| log(Cmax)* | 1.031 | 1.855 |
| log(AUClast)* | 1.037 | 1.540 |
| log(AUCinf)* | 1.050 | 1.461 |
| log(normCmax)* | 0.897 | 1.589 |
| log(normAUClast)* | 0.921 | 1.290 |
| log(normAUCinf)* | 0.937 | 1.220 |

*Note:
the actual confidence limits obtained for these parameters have been exponentiated since the data were log-transformed originally.

Since none of these confidence intervals for the non-standardized data are contained in the interval from 0.8 to 1.25, the conclusion is that the two sprayers are not equivalent when compared on raw values. For Tmax, the one-sided t-test for H₀: Test/Reference<0.8 is not rejected. Also, the tests of H₀: Test/Reference>1.25 are not rejected for any of the log-transformed raw values. While the normalization by dispensed doses does improve the comparability of the two delivery systems, two of the three parameters fail to reject the null hypothesis H₀: Test/Reference>1.25. Bioequivalence is supported only by the pair of one-sided tests for the normalized, log-transformed AUC(inf). Both one-sided t-test for each of the seven parameters have been performed at an alpha level of 0.05.

The data shows a remarkably high degree of non-bioequivalence for an FDA-approved system that has been sold and dispensed for a number of years. The degree of non-equivalence is also significantly greater than that of the method of the invention using the Pfeiffer device. Based on the greater consistency among individual doses uses the system of the invention, the small excess in unit-dose administration can be further reduced by adjusting the volume of, and/or drug concentration in the liquid therapeutic composition placed in the delivery device.

Equality of Variances

The Pitman-Morgan adjusted F test was used to compare variances of the unit-dose and multi-dose parameters. (See Chow, S -C. and Liu, J -P, Design and Analysis of Bioavailability and Bioequivalence Studies. Marcel Dekker, inc., New York (2000)). Since this test could not be generalized to the three period design, the first two periods of the butorphanol trial were used, and for the purposes of this analysis, there are two formulations, two periods, and two sequences. The Pitman-Morgan adjusted F test can be used even if the period effect is significant, and has a simplified form in the absence of period effects. Of the seven PK parameters considered, only Tmax exhibited a significant period effect. Table 3 summarizes the results of the tests of equality. The null hypothesis is that the variances are equal, and small p-values are indicative of a departure from equality.

TABLE 3
Summary of the Pitman-Morgan's adjusted F tests
for PK parameters

| Parameter | Pitman-Morgan F value | p-value |
|---|---|---|
| Tmax | 0.3 | 0.6 |
| log(Cmax) | 11.3 | 0.005 |
| log(AUClast) | 30.1 | <0.0001 |
| log(AUCinf) | 15.3 | 0.002 |
| log(normCmax) | 8.4 | 0.01 |
| log(normAUClast) | 23.7 | 0.0002 |
| log(normAUCinf) | 10.7 | 0.005 |

The tests of equality variances indicate that for all PK parameters except Tmax, the variabilities of the two formulations are significantly different, with the unit dose system demonstrating much lower variability of drug levels in the blood. While the normalization of the Cmax, AUC(last) and AUC(inf) parameters somewhat decreased the difference between the variances (as evidenced by slightly smaller F values), the variances were nonetheless significantly different. The variability associated with the unit-dose system was smaller than that of the multi-dose system of the prior art, which is consistent with the findings of the delivery volume weight study.

From the above, it is apparent that the dose weight/volume data is confirmed by the blood level (pharmacokinetic) analysis. The prior art delivery system results in an area under the curve that is 90% of the delivery system of the present invention. This difference is highly significant from a patient therapy standpoint. When FDA-prescribed bioequivalence statistical methods are applied, it is concluded that the products as administered to the patients are not equivalent. Thus, the method and system of the invention provide an unexpected improvement in the intranasal administration of butorphanol.

As will be understood by one of ordinary skill in the art, the results and conclusions drawn above from the study of the intranasal administration of butorphanol can be extended in the practice of the invention to other opioids that have been approved for intranasal administration in the form of a liquid spray using commercial applicators of the type utilized in the comparison study. As will also be comprehended by those workers possessed of ordinary skill in the art from the examples and data that follow, the method and system of the invention can be practiced to the advantage and benefit of patients, of medical facilities and medical professionals, and of society at large for the intranasal administration of other opioids and controlled substances.

Hydromorphone Intranasal Solution

In accordance with the methods and apparatus described above, hydromorphone HCl (dihydromorphinone hydrochloride) was formulated in a liquid composition for use in the practice of the invention. Hydromorphone HCl (“HM HCl”) is a potent mu-receptor agonist opiate analgesic with properties similar to morphine. HM HCl is chemically similar to morphine, oxymorphone, and codeine and shares many of their analgesic and pharmacological properties. HM HCl is a prescription drug narcotic analgesic, more commonly known by the trade name of DILAUDID® (Merck Index, 1983). Dilaudid (C₁₇H₁₉O₃N.H₂O) was discovered by the A. G. Knoll chemical firm of Ludwigshafen, Germany and was the subject of a 1923 patent. The first literature describing the synthesis and testing of this medication appeared in the 1920's and it has been used in the clinical management of pain since then. The first extensive literature review was published in 1933 by the Council on Pharmacy and Chemistry in the Journal of the American Medical Association (Eddy, N B. Dilaudid (Dihydromorphoninone hydrochloride) J Am Med Assoc 1933;100: 1032-1035). The drug is approved and widely accepted in the medical community as a safe and effective analgesic. It is presently marketed under the trade name Dilaudid® and Dilaudid-HP by Knoll Pharmaceutical Company.

It is known that HM HCl is subject to hepatic first pass metabolism when administered orally or by suppository. Thus, when administered intranasally, the effective unit-dose can be substantially less as compared to doses administered by oral or rectal routes.

The HM HCl is preferably prepared in the form of a single or unit-dose nasal spray for intranasal administration by a precision dosage manually activated pump. Each 1 ml of nasal spray solution is preferably formulated to contain 10 mg HM hydrochloride with 0.2% sodium citrate, 0.2% citric acid solution, and sterile (i.e., water for injection, USP), accepted antioxidant concentration and buffer in pharmaceutical products.

As will be understood by those familiar with the art, dosage forms at lower concentrations of HM can be prepared for administration based upon the patient's lower body weight, as in the case of children or adults of substantially smaller size. The nasal spray solution has a pH in the range of from about 3 to about 7, with a pH of about 5 being preferred.

In a preferred delivery system, each actuation of the nasal spray pump delivers 0.1 ml of this 10 mg/ml HM HCl solution constituting a 1 mg dose. A smaller dose may be administered to children.

The filled applicators can be sterilized by methods well known in the art. The HM HCl nasal spray applicators are stored at 15°-30° C. (59°-86° F.) and protected from light to provide for maximum shelf life. Since the applicator body is not transparent, visual inspection of the drug product for signs of deterioration is not possible and attention to the expiration date and storage conditions is important. Any expired product is discarded in the appropriate manner.

An analysis of previous work describing intranasal (IN) administration of narcotics suggested that HM HCl is highly likely to have good bioavailability by the IN route in view of its potency and water solubility. Extensive review of hydromorphone literature did not reveal any comparative IV/IM/IN concentration versus time or pharmacokinetic data. A protocol was designed to determine the bioavailabity of HM HCl by the IM and IN routes by comparing the pharmacokinetics of intramuscularly administered HM HCl and intranasally administered HM HCl to HM HCl administered via the IV route. Specifically, the objectives of this study were: (1) to compare the pharmacokinetics of HM via intranasal, intramuscular, and intravenous administration of a 2 mg dose of HM HCl; and (2) to evaluate the bioavailability of 2 mg HM HCl after intranasal, IM and IV routes of administration using a standard three-period, crossover design.

A formulation of HM HCl for intranasal administration was prepared in the form of a liquid composition at a concentration of 1.0 mg of HM HCl in 0.1 mL. The composition was used to fill the required number of single-dose, metered sprayers commercially produced and sold by Pfeiffer of America, Inc. Each subject received a single spray in each nostril for a total of 2.0 mg. A 2.0 mg dose is preferred as being within common, safe and labeled doses prescribed for pain management. Commercially available HM HCl (Dilaudid® for parental administration from Knoll Pharmaceutical Company) was purchased for IM/IV administration.

Investigational Methods

Nine healthy male subjects between the ages of 22 and 28 years participated in this inpatient study. Study participants were selected based on inclusion/exclusion criteria, history and physical exam, laboratory tests, and other customary procedures.

Subject demographics were recorded. These included age range: 22-28 years; height range: 175-188 cm; weight range: 70.3-95.3/kg; origin: six Caucasian, two Asian, one Native American; all were non-smokers.

All nine of the subjects completed the study according to the protocol. Each of the subjects received 3 doses of 2 mg of HM HCl on three separate occasions. No clinically significant protocol violations occurred during this study. Because the inclusion criteria mentioned abstinence from prescription and non-prescription drugs prior to and during the study, any medications taken in the 14 days before the study and during the study were noted.

Clinical Trials

Study Drug Formulation

HM HCl for intranasal administration was supplied by the University of Kentucky College of Pharmacology. HM HCl for intravenous administration was supplied as Dilaudid® 1 mg/mL for subjects 1, 3, 8, and 9 on the first day and for subjects 2, 4, 5, 6, 7 on the second study day. HM HCl for intramuscular administration was supplied as Dilaudid® 4 mg/mL for subjects 2, 4, 5, 6 and 7 on first study day and for subjects 1, 3, 8 and 9 on the second study day. Free base content was 1.77 mg or 88.7% of stated HM HCl strength (from molecular weights: 321.8−36.46=285.34, 285.34/321.8=88.7%) To summarize, the dosages for each of the three routes of administration were as follows:

Treatment A: 2.0 mg intravenous HM HCl;

Treatment B: 2.0 mg intramuscular HM HCl; and

Treatment C: 2.0 mg intranasal HM HCl solution

Study Drug Administration

On days 1 and 8, 2.0 mg of HM HCl was given intravenously or intramuscularly in random order following an overnight fast. On day 15, 3.0 mg of HM HCl was given intranasally following an overnight fast (except for water ad lib). Subjects were not permitted to recline for 4 hours following drug administration and remained fasting for 4 hours (until lunch) on these study days.

Meals and snacks prepared by the University of Kentucky Hospital Dietetics and Nutrition department were provided for each subject. Subjects were instructed to eat all of their meals. All subjects received identical meals and snacks on each of the treatment days, but received different meals on the different study days.

Safety Measures

Weight, blood pressure, and pulse were measured prior to dosing and at the end of the study. Blood pressure and pulse rate were measured with the subjects seated in an upright position before any corresponding blood sample was collected. Blood pressure and pulse rate were measured and recorded on the same arm throughout the study at 0 (pre-dose) and 30 minutes, 1, 2, 4, 8, and 16 hours.

Clinical Adverse Events

Spontaneously reported adverse events were recorded by the subjects throughout the study; adverse events were also elicited by nondirected interviews.

Sample Collection

Blood samples for period I through period III were collected from each subject according to the following schedule: 0 (pre-dose), 5, 10, 15, 20, 30 and 45 minutes, and 1, 2, 3, 4, 6, 8, 12 and 16 hours following HM HCl administration. The beginning of the IV administration was considered time zero. After collection, the blood was centrifuged in a refrigerated centrifuge at 4° C. to separate the plasma and the cells, and the plasma was transferred to polypropylene tubes. The plasma was stored at approximately −70° C. at the study site until shipped to an independent analytical service. The plasma was maintained frozen during shipping and upon arrival at the remote analytical facility, the samples were stored at approximately −20° C. until analyzed.

Bioanalytical Methods

LC/MS/MS Assay for Hydromorphone

The sample analysis was performed by an independent service in accordance with established protocols. Concentrations less than 20 pg/mL were reported as below quantitation limit (BQL). Samples with concentrations greater than 2,000 pg/mL were reanalyzed using a dilution so that the assayed concentration was within the range of 20 to 2,000 pg/mL. QC samples were also diluted. During the validation, the precision was expressed as the percent coefficient of variation (% CV) and the accuracy as the percent difference from the theoretical (same as relative error).

Pharmacokinetic Methods

Plasma concentration versus time date for HM were analyzed using noncompartmental pharmacokinetic methods.

Maximum plasma concentration (Cₘₐₓ) and the corresponding sampling time (Tₘₐₓ) were identified by observation. Concentration versus time data were plotted on a semi-logarithmic scale and the terminal log-linear phase was identified by visual inspection. The elimination rate constant (λ_{z}) was determined as the slope of the linear regression for the terminal log-linear portion of the concentration versus time curve. The terminal half-life value (t_{1/2}) was calculated as 0.693 divided by λ_{z}.

The area under the curve plotting plasma concentration versus curve (AUC) was calculated by the trapezoidal rule and extrapolated to infinite time. The AUC to the last time point (AUC₀₋ₗₐₛₜ) was computed by the linear trapezoidal rule. Mean plasma concentration were calculated for graphical presentation only. Data included in the mean calculation were for samples with measurable concentrations drawn within 5% of the nominal sampling time.

Safety Results

Results of the clinical measurement of vital signs and body weight exams were recorded and nasal exams were performed. A review of this data failed to reveal any clinically significant safety concerns. There were no serious adverse events and no subjects were discontinued due to adverse effects. Subjects commented that the intensity of the drug effects were lower with the IN route compared to the IV or IM administrations.

Bioanalytical Results

Hydromorphone in Plasma by LC/MS/MS

Results from the control samples and calibration curves analyzed with the study samples and the method validation was reported. The overall CV which reflects precision was <7.4% for the QC samples. The percent recovery ranged from 94.5 to 100.1% for QC concentrations 200.0, 500.0, and 1000 which reflects accuracy was <6% for the QC samples.

Pharmacokinetic Results

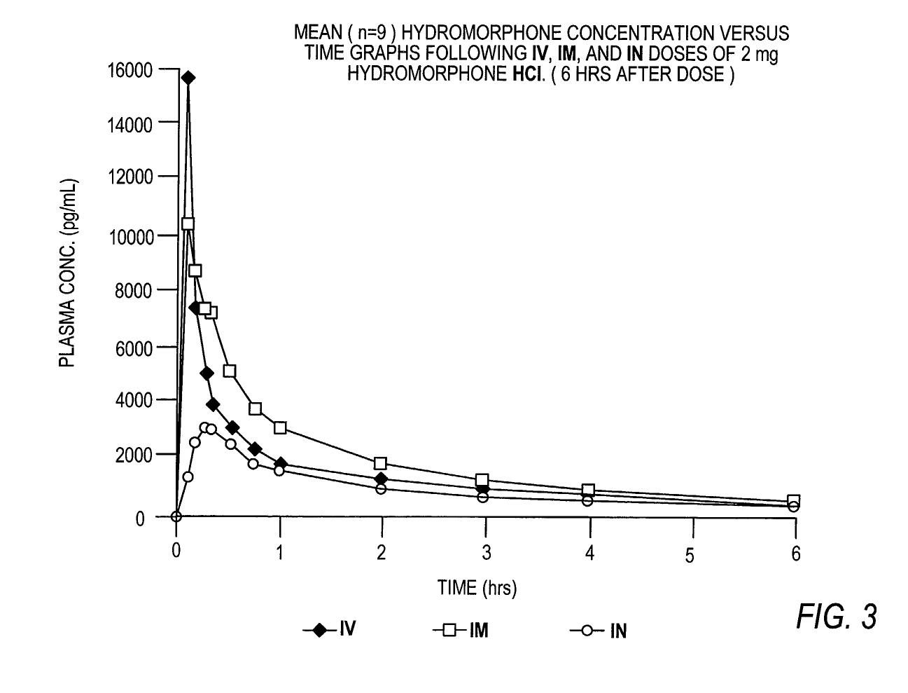

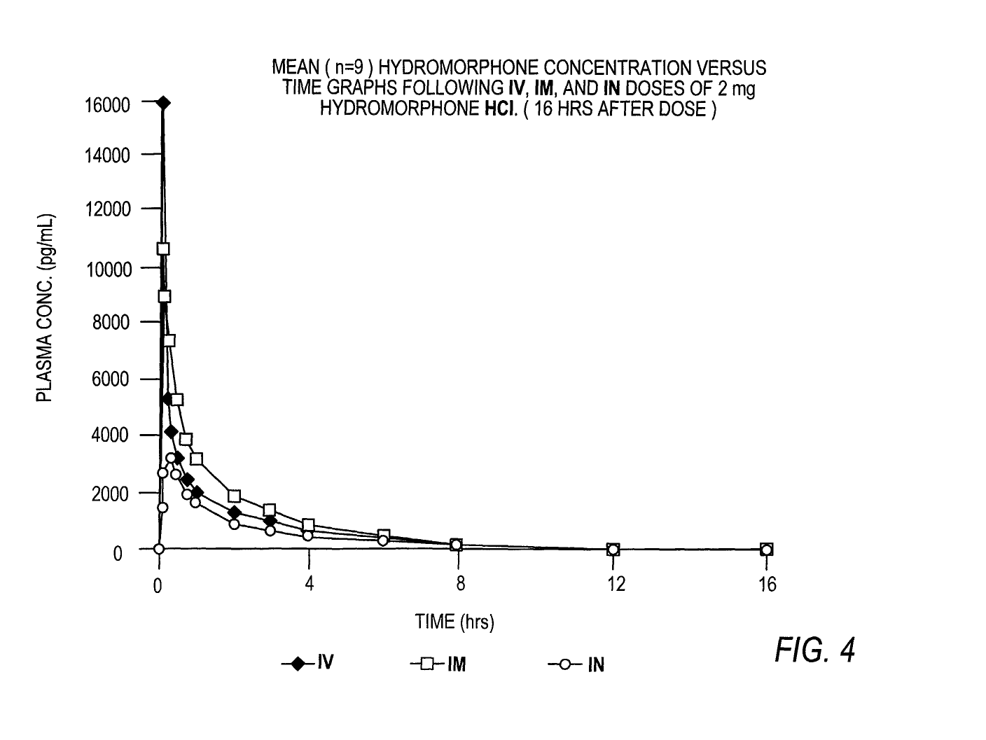

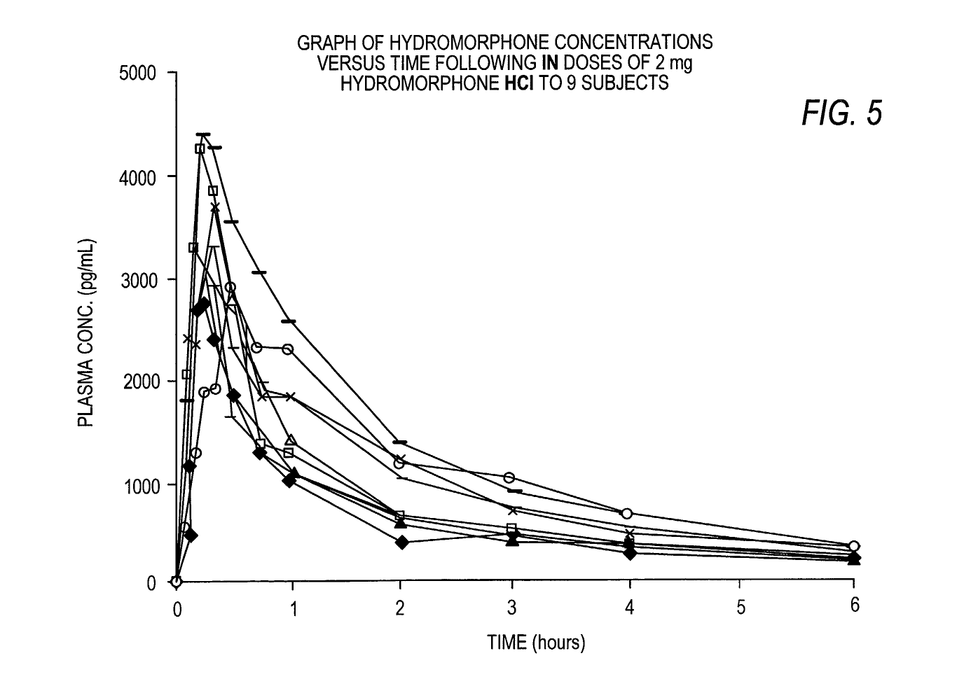

The plasma HM HCl concentrations and actual collection times for each of the 9 subjects was tabulated and plasma concentration-time curves for each of the 9 subjects were prepared. Mean concentration-time curves of FIGS. 3 and 4 are representative for most subjects (mean data tabulation). FIG. 3 is a plot of the mean (n=9) hydromorphone concentration versus time graphs following IV, IM and IN doses of 2 mg hydromorphone HCl during the 6 hours after dose; FIG. 4 is the same data plotted for 16 hours after the dose. Curves for all subjects for 6 hours after the IN dose appear in FIG. 5 as a graph of hydromorphone concentrations versus time following IN doses of 2 mg hydromorphone HCl to 9 subjects.

Noncompartmental pharmacokinetic analysis was used to evaluate the plasma concentration versus time curves of HM following single 2.0 mg doses of HM HCl by intravenous (IV), intramuscular (IM), and intranasal (IN) routes. Individual plasma HM concentrations versus time profiles for all subjects were recorded; the number of time points used to estimate the elimination rate constant were also recorded; and a complete listing of individual and mean pharmacokinetic parameters for all 9 subjects was recorded. Table 4.2 is a summary of the descriptive statistics for HM pharmacokinetic parameters.

Rapid absorption of HM HCl was observed after the IM and IN doses. The Tₘₐₓ values were approximately 9 and 18 minutes, on average, for the IM and IN doses, respectively. The mean Tₘₐₓ for the IV infusion was not the first blood sample after the end of the infusion for two reasons. The peak concentration after the IV dose in one subject was not at the first blood sample after the end of the IV infusion, but at the next time point. In the case of Subject 4, acquiring the blood sample immediately following the IV infusion was delayed resulting in the mean Tₘₐₓ being affected. As expected, the HM Cₘₐₓ and AUCs were significantly higher after IM and IV administration compared to IN administration. Mean plasma half-lives and clearance (after correcting for bioavailability) were similar for all three treatments.

The arithmetic mean value of absolute bioavailability of HM from the IN formulation is 64%. The range was 50% to 81% bioavailability compared to the IV dose. The apparent bioavailability of the IM HM HCl was about 30% greater than that of the same dose of IV administration. The source of this aberrant phenomenon was not found, but unusual distribution phenomena after parenteral administration have been reported by others working in this field.

Statistical Evaluation

The pharmacokinetic parameters in Table 4.3 were analyzed to evaluate the effect of routes of administration and to test for period and sequence effects. The analysis of this pilot data is considered in two parts: the first part considers only the first two periods and includes the factors of treatment, sequence (i.e., a test of carryover effects) and period; the second part contains all three periods and treatments, but ignores the effects of sequence and period. The 2-period analysis is noted in Table 4.3 as period 1 vs. 2 and the last column contains the 3-period model.

There are even more significant treatment effects for these nine outcomes. Post-hoc analyses are based on Fisher's least significant difference procedure and displayed in Table 4.3. In light of the fact that there were no significant period or sequence effects (using an alpha level of 0.05), and since this is a pilot project, it is arguable that the above analysis is appropriate.

Since the Cₘₐₓ value for Subject 07 was beyond 2 standard deviations of the mean with all measurements included, there is an objective method for omitting this value for this subject. Analyses with and without this outlier gave the same result.

TABLE 4
Summary of significance levels from IN
2-period and 3-period model

| | Sequence | Period | Treatment: | Treatment: |
|---|---|---|---|---|
| Parameter | (1 vs 2) | (1 vs 2) | IV vs IM | (IV vs IM vs IN) |
| Tₘₐₓ | NS* | NS | NS | .0001 |
| Cₘₐₓ | NS | .032 | .071 | .0001 |
| Cₘₐₓ (omit outlier) | NS | .062 | NS | .0001 |
| AUC₀₋ₜ | NS | NS | .0001 | .0001 |
| AUC_{0-∞} | NS | NS | .0001 | .0001 |
| t_{1/2} | NS | NS | NS | NS |
| CL/F | NS | NS | .0001 | .0001 |
| Dose | NS | NS | .0001 | .0001 |
| λ_{Z} | NS | NS | NS | NS |

*All p-values reported as ‘NS’ are >0.1.

In this study of nine healthy male subjects that received 2 mg hydromorphone HCl by IV, IM and IN routes, comparisons between the IM and IN doses for purposes of bioequivalence could not be completed when it was found that the hydromorphone concentrations for the IM dose were markedly different as compared to those from the IN doses.

Noncompartmental analysis of the pharmacokinetic data gave results similar to previous studies with respect to half-lives, clearance, rapid distribution into the tissues, and large apparent distribution volume (Parab et al. 1988; Hill et al. 1991), although comparisons between this study and previous studies should be done with caution because of differences in analytical techniques. Hydromorphone HM HCl is well absorbed by the nasal route. Intranasal bioavailability was approximately 64%, on average. Interindividual variation was smaller for Cₘₐₓ and Tₘₐₓ for the IN route compared to the IV and IM routes. Three compartment characteristics were suggested by the tri-phasic concentration versus time curves, but compartmental analysis was not performed.

After the short IV infusion, the hydromorphone concentrations peaked at the end of the infusion as expected in all but one subject. Peak concentrations after the IM dose were unexpectedly rapid and precluded the analysis of the data for showing the bioequivalence of the IM and IN doses, and that analysis was not pursued.

Pharmacokinetic parameter estimates yielded CVs less than 27% for IN pharmacokinctic parameters except for Vₛₛ (CV 46%). Estimates of within-subject variability were smaller than estimates for published studies of IV HM HCl (Parab et al.; Hill et al.; Vallner et al.). Using a crossover design and standardizing meal times in this study likely helped to lower within-subject variability.

Clearance is similar for all three routes of administration regardless of route. Variabilities in CL and Vₛₛ estimates are less after the IV dose compared to the IN dose The reduced variability is expected since IV dosing avoids between-subject variability in absorption and first-pass metabolism.

Adverse events were less frequent and milder after the IN dose compared to the IV and IM doses. Assuming a dose-response relationship, this effect believed to be attributable to the fact that the bioavailability of the IN dose was less and the peak concentration lower, so the subjects effectively received a lower dose that was more slowly absorbed. Nasal irritation was not observed with the exception of a bad taste in the throat reported by most subjects after the IN dose. In summary, HM HCl is well absorbed by the nasal route with bioavailability of 64%. Cmax and Tmax were similar for IM and IV routes. Clearance is similar regardless of route.

HM HCl produced no systemic adverse events beyond those commonly experienced by injection. After single IN doses the subjects complained of bitter taste as the only local administration effect of the formulation. Detailed nasal examination demonstrated no pathology of the naso-pharynx after single administration of the HM HCl formulations.

In a further series of studies, HM HCl is administered in accordance with the method of the invention as described above to larger groups of volunteers selected from the following categories:

1. in good health—ages 18 to 40;

2. in good health—ages 60 to 80;

3. patients with rhinitis;

4. post-partum breast feeding for milk transfer;

5. post-operative pain in women;

6. children and adolescents with cancer;

7. male knee surgery patients; and

8. male and female surgical patients.

The results of these studies indicate the HM HCl is suitable for use in providing relief from pain in a wide variety of settings without adverse side effects that are any more significant than those reported for the alternate routes of administration, and provides the advantages of convenience, rapid onset.

Liquid formulations are prepared as fully dissolved solutions in a nasal carrier of each of the following systemic analgesics: morphine, apomorphine, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl.

Clinical testing of each of the above liquid compositions in accordance with the method of the invention as practiced in the hydromorphone HCl clinical test using a Pfeiffer unit-dose applicator produces results comparable to those obtained in the hydromorphone HCl work.

What is claimed is:

1. A pharmaceutical drug dosage delivery system for intranasal administration, as to a warm-blooded animal, of a predetermined dosage of a pharmaceutically active agent that has been approved for use in producing a pharmacologically induced physiological response in the animal, the pharmaceutical drug dosage delivery system comprising:
a. at least one unit-dosage of the pharmaceutically active agent, selected from the group consisting of: morphine, apomorphine, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, for delivery by intranasal administration as a liquid spray, each unit-dosage having a total volume and containing:
i. an effective amount of the pharmaceutically active agent for inducing the desired physiological response, the pharmaceutically active agent being present as a solute of a liquid solution; and
ii. a volume of a physiologically acceptable solvent-carrier for each unit-dosage of the pharmaceutically active agent solute, the solvent-carrier being selected on the basis of the solubility of the pharmaceutically active agent solute in the solvent-carrier;
such that the liquid solution of the pharmaceutically active agent in the physiologically acceptable solvent-carrier has a pre-determined concentration, whereby a pre-determined volume, of from about 0.025 ml to about 0.75 ml, of the liquid solution, contains at least one unit-dosage of the effective amount of the pharmaceutically active agent;
b. at least one container for containing the volume of liquid solution [of the at least one unit-dosage of the pharmaceutically active agent dissolved in the physiologically acceptable solvent-carrier, that provides the at least one unit-dosage of the effective amount of the pharmaceutically active agent upon intranasal administration of the dosage, as the contents of the container, with the container having, and the contents therein being under a seal;
c. at least one dispensing applicator, into which the at least one container is inserted, the dispensing applicator having means for breaking the seal of the container, means for forming a spray of the volume of liquid solution that is in the container, and means for delivering at least 97% by volume and not more than 103% by volume of the predetermined dosage into a nasal cavity as a liquid spray, upon breaking of the seal of the container, and such that there is essentially no significant quantity of the therapeutic composition containing the pharmaceutically active agent remaining in the container or the dispensing applicator after application.

2. The pharmaceutical drug dosage delivery system according to claim 1, wherein the liquid spray is an atomized mist.

3. The pharmaceutical drug dosage delivery system according to claim 1, wherein the liquid spray is an aerosol.

4. The pharmaceutical drug dosage delivery system according to claim 1, wherein the container and the dispensing applicator contains no recoverable quantity of pharmaceutically active agent.

5. The pharmaceutical drug dosage delivery system according to claim 1, wherein the amount of the pharmaceutically active agent that is contained in a unit dosage as the effective amount of the pharmaceutically active agent, is an amount of the pharmaceutically active agent determined to produce a desired level of bio-availability of the pharmaceutically active agent in the animal in a predetermined time after administration of the unit dosage.

6. The pharmaceutical drug dosage delivery system according to claim 1 comprising one dispensing applicator.

7. The pharmaceutical drug dosage delivery system of claim 8 wherein the volume of liquid solution is equivalent to two unit-doses.

8. The pharmaceutical drug dosage delivery system according to claim 1 wherein the pharmaceutically active agent is hydromorphone.

9. The pharmaceutical drug dosage delivery system according to claim 1, wherein the pharmaceutically active agent is butorphanol.

10. The pharmaceutical drug dosage delivery system according to claim 1, wherein the pharmaceutically active agent is butorphanol; the non-toxic, physiologically acceptable solvent-carrier is water; the liquid solution of butorphanol in water has a concentration of about 10 mg/ml; the liquid solution is pH adjusted to a pH of about 5; the effective amount of butorphanol capable of being intranasally delivered by administration of the dosage is from about 1 to 2 mg; the unit-dosage volume is from about 0.1 ml to about 0.2 ml; and the container has a single chamber containing a single unit dose having a sealed liquid solution volume of from about 0.1 ml to about 0.2 ml.

11. The pharmaceutical drug dosage delivery system according to claim 1, wherein the pharmaceutically active agent is hydromorphone; the non-toxic, physiologically acceptable solvent-carrier is water; the liquid solution of butorphanol in water has a concentration of about 10 mg/ml; the liquid solution is pH adjusted to a pH of about 5; the effective amount of butorphanol capable of being intranasally delivered by administration of the dosage is from about 1 to 2 mg; the unit-dosage volume is from about 0.1 ml to about 0.2 ml; and the container has a single chamber containing a single unit dose having a sealed liquid solution volume of from about 0.1 ml to about 0.2 ml.

12. A drug dosage storage and delivery system for providing a precisely measured dosage of a drug to a patient by intranasal administration thereto of the drug in the form of a liquid spray, the system comprising:
a. a dosage unit containing at least one unit-dosage of a pharmaceutically active agent, selected from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, in liquid solution form, for delivery by intranasal administration to the patient, as an atomized liquid spray, the dosage unit having a volume which is a total volume of all unit-dosages contained in the dosage unit, such that each unit-dosage of the dosage unit contains:
i. an effective amount of the pharmaceutically active agent, sufficient to induce a physiological response, the pharmaceutically active agent being present as a solute of a liquid solution; and
ii. a volume of a physiologically acceptable solvent-carrier for the pharmaceutically active agent solute, the solvent-carrier being selected on the basis of the solubility of the pharmaceutically active agent solute in the solvent-carrier;
such that the liquid solution of the pharmaceutically active agent in the physiologically acceptable solvent-carrier has a pre-determined concentration, whereby a volume of the liquid solution, of from about 0.025 ml to about 0.75 ml, contains one unit-dosage of the effective amount of the pharmaceutically active agent;
b. a container having at least one liquid storage compartment, for containing the volume of liquid solution of the at least one unit-dosage of the pharmaceutically active agent dissolved in the physiologically acceptable solvent-carrier, that delivers at least one unit-dosage of the effective amount of the pharmaceutically active agent upon administration of the dosage, as the contents of the compartment, with the container having, and the contents therein being under, a breakable seal;
c. a metered dispensing applicator of the intranasal drug delivery device, into which the container is inserted, the metered dispensing applicator having means for breaking the seal of the container, means for forming a spray of the volume of liquid solution that is in the container, and means for delivering at least 97% by volume and not more than 103% by volume of the predetermined dosage into a nasal cavity as a liquid spray, upon breaking of the seal of the container.

13. A single-dose, single-use hydromorphone unit-dosage intranasal drug dosage delivery system comprising:
a. a single unit-dosage of hydromorphone, in liquid solution form, for delivery by intranasal administration to a person, as a liquid spray, each unit-dosage having a total volume not greater than about 2 ml, and containing:
i. an effective amount of hydromorphone, of from about 0.25 mg to about 10.0 mg, for inducing a systemic analgesic physiological response in the person, and to produce a desired level of bio-availability of the hydromorphone after administration, the hydromorphone being present as a solute of a liquid solution; and
ii. a volume of a physiologically acceptable solvent-carrier for the unit-dosage of the hydromorphone, the solvent-carrier being selected on the basis of the solubility of the hydromorphone therein;
such that the liquid solution of the hydromorphone in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the hydromorphone;
b. a disposable container for use in an intranasal drug delivery device, for containing the volume of liquid solution of the one unit-dosage of the hydromorphone dissolved in the physiologically acceptable solvent-carrier, that provides the one unit-dosage of the effective amount of the hydromorphone upon intranasal administration of the dosage, as the contents of the container, with the container having, and the contents therein being under, a breakable seal;
c. a disposable metered applicator of the intranasal drug delivery device, into which the container is inserted, the metered applicator having means for breaking the seal of the container, means for forming a spray of the volume of liquid solution that is in the container, and means for delivering at least 97% by volume and not more than 103% by volume of the predetermined dosage into a nasal cavity as a liquid spray, upon breaking of the seal of the container.

14. A single-dose, single-use butorphanol unit-dosage intranasal drug dosage delivery system comprising:
a. a single unit-dosage of butorphanol, in liquid solution form, for delivery by intranasal administration to a person, as a liquid spray, each unit-dosage having a total volume not greater than about 2 ml, and containing:
i. an effective amount of butorphanol, of from about 0.25 mg to about 10.0 mg, for inducing a systemic analgesic physiological response in the person, and to produce a desired level of bio-availability of the butorphanol after administration, the butorphanol being present as a solute of a liquid solution; and
ii. a volume of a physiologically acceptable solvent-carrier for the unit-dosage of the butorphanol, the solvent-carrier being selected on the basis of the solubility of the butorphanol therein;
such that the liquid solution of the butorphanol in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the butorphanol;
b. a disposable container for use in an intranasal drug delivery device, for containing the volume of liquid solution of the one unit-dosage of the butorphanol dissolved in the physiologically acceptable solvent-carrier, that provides the one unit-dosage of the effective amount of the butorphanol upon intranasal administration of the dosage, as the contents of the container, with the container having, and the contents therein being under, a breakable seal;
c. a disposable metered applicator of the intranasal drug delivery device, into which the container is inserted, the metered applicator having means for breaking the seal of the container, means for forming a spray of the volume of liquid solution that is in the container, and means for delivering at least 97% by volume and not more than 103% by volume of the predetermined dosage into a nasal cavity as a liquid spray, upon breaking of the seal of the container.

15. A method for the intranasal administration of at least one pre-measured volume of a predetermined dosage amount of a pharmaceutically active agent, as a liquid spray, to at least one warm-blooded animal, for the purpose of producing a pharmacologically induced physiological response in the animal, the method comprising the steps of:
a. selecting a pharmaceutically active agent, selected from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, that has been approved for use in producing the desired response, for use as a solute of a liquid solution;
b. determining an effective dosage amount of the pharmaceutically active agent for delivery by intranasal administration so as to produce a desired level of bio-availability of the pharmaceutically active agent in the animal, after administration;
c. determining a physiologically acceptable solvent-carrier for the pharmaceutically active agent solute, such that a volume of liquid solution of the pharmaceutically active agent in the physiologically acceptable solvent-carrier, which contains the effective dosage amount of the pharmaceutically active agent, is not greater than from about 0.025 ml to about 0.75 ml;
d. forming a liquid solution of the pharmaceutically active agent dissolved in the physiologically acceptable solvent-carrier, with the liquid solution having a pre-determined concentration, such that a pre-determined volume of the liquid solution contains at least one unit-dosage of the effective amount of the pharmaceutically active agent;
e. placing a volume of liquid solution of the pharmaceutically active agent dissolved in the physiologically acceptable solvent-carrier, sufficient to provide at least one unit-dosage of the effective amount of the pharmaceutically active agent, in at least one container for use in an intranasal drug delivery device;
f. sealing the container with the contents therein under a breakable seal;
g. making the container available for insertion into and use together with at least one precisely metered dispensing applicator of a intranasal drug delivery system; and
h. intranasally administering the effective dosage amount of the pharmaceutically active agent dissolved in the physiologically acceptable solvent-carrier by breaking the seal of the container with seal-breaking means of the dispensing applicator to release the contents of the container through an outlet opening of the dispensing applicator, as a liquid spray, into a nasal cavity.

16. A method for the intranasal administration of at least one pre-measured volume of a predetermined dosage amount of a pharmaceutically active agent, as a liquid spray, to at least one warm-blooded animal, for the purpose of producing a pharmacologically induced physiological response in the animal, the method comprising the steps of:
a. preparing a dosage unit containing at least one unit-dosage of a pharmaceutically active agent, selected from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, in liquid solution form, for delivery by intranasal administration to the patient, as a liquid spray, the dosage unit having a volume which is a total volume of all unit-dosages contained in the dosage unit, such that each unit-dosage of the dosage unit contains:
i. an effective amount of the pharmaceutically active agent sufficient to induce a physiological response, the pharmaceutically active agent being present as a solute of a liquid solution; and
ii. a volume of a physiologically acceptable solvent-carrier for the pharmaceutically active agent solute, the solvent-carrier being selected on the basis of the solubility of the pharmaceutically active agent solute in the solvent-carrier;
such that the liquid solution of the pharmaceutically active agent in the physiologically acceptable solvent-carrier has a pre-determined concentration, whereby a volume of the liquid solution, of from about 0.025 ml to about 0.75 ml, contains one unit-dosage of the effective amount of the pharmaceutically active agent;
b. providing a container having at least one liquid storage compartment, for containing the volume of liquid solution of the at least one unit-dosage of the pharmaceutically active agent dissolved in the physiologically acceptable solvent-carrier, as the contents of the compartment, with the container having, and the contents therein being under, a breakable seal;
c. providing a metered dispensing applicator of the intranasal drug delivery device, into which the container is inserted, the metered dispensing applicator having means for breaking the seal of the container, means for forming a spray of the volume of liquid solution that is in the container, and means for delivering at least 97% by volume and not more than 103% by volume the contents of the compartment into a nasal cavity as a liquid spray, upon breaking of the seal of the container.

17. A method for intranasal administration to a person, of a single unit-dose of hydromorphone, as a liquid spray, utilizing a single unit-dosage sized container of hydromorphone, the container being disposable after use, and utilizing a metered drug delivery device that is disposable after use, the method comprising the steps of:
a. providing a single unit-dosage of hydromorphone, in liquid solution form, for delivery by intranasal administration to a person, as an atomized liquid spray, each unit-dosage having a total volume not greater than about 2 ml, and containing:
i. an effective amount of hydromorphone, of from about 0.25 mg to about 10.0 mg, for inducing a systemic analgesic physiological response in the person, and to produce a desired level of bio-availability of the hydromorphone after administration, the hydromorphone being present as a solute of a liquid solution; and
ii. a volume of a physiologically acceptable solvent-carrier for the unit-dosage of the hydromorphone, the solvent-carrier being selected on the basis of the solubility of the hydromorphone therein;
such that the liquid solution of the hydromorphone in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the hydromorphone;
b. providing a disposable container having at least one liquid storage compartment, for use in an intranasal drug delivery device, for containing the volume of liquid solution of the one unit-dosage of the hydromorphone dissolved in the physiologically acceptable solvent-carrier, with the container having, and the contents therein being under, a breakable seal;
c. providing a disposable metered applicator of the intranasal drug delivery device, into which the container is inserted, the metered dispensing applicator having seal-breaking means for breaking the seal of the container, spraying means for forming a spray of the volume of liquid solution that is in the container, and delivery means for delivering at least 97% by volume and not more than 103% by volume of the predetermined dosage into a nasal cavity as a liquid spray, upon breaking of the seal of the container such that there is essentially no significant quantity of the therapeutic composition containing the pharmaceutically active agent remaining in the container or the dispensing applicator after application;
d. assembling the intranasal drug delivery device by inserting the container into the metered dispensing applicator;
e. inserting a contoured head portion of the metered applicator into a nasal cavity;
f. breaking the seal of the container with the seal-breaking means;
g. simultaneously withdrawing the liquid solution contents of the container and forming a liquid spray thereof with the spraying means; and
h. delivering the liquid spray through the delivery means to an outlet opening in the head portion of the metered dispensing applicator, which is in communication with the delivery means, from which the liquid spray, containing the unit-dosage of hydromorphone, is released into the nasal cavity.

18. A method for intranasal administration to a person, of a single unit-dose of butorphanol, as a liquid spray, utilizing a single unit-dosage sized container of butorphanol, the container being disposable after use, and utilizing a metered drug delivery device that is disposable after use, the method comprising the steps of:
a. providing a single unit-dosage of butorphanol, in liquid solution form, for delivery by intranasal administration to a person, as an atomized liquid spray, each unit-dosage having a total volume not greater than about 2 ml, and containing:
i. an effective amount of butorphanol, of from about 0.25 mg to about 10.0 mg, for inducing a systemic analgesic physiological response in the person, and to produce a desired level of bio-availability of the butorphanol after administration, the butorphanol being present as a solute of a liquid solution; and
ii. a volume of a physiologically acceptable solvent-carrier for the unit-dosage of the butorphanol, the solvent-carrier being selected on the basis of the solubility of the butorphanol therein;
such that the liquid solution of the butorphanol in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the butorphanol;
b. providing a disposable container having at least one liquid storage compartment, for use in an intranasal drug delivery device, for containing the volume of liquid solution of the one unit-dosage of the butorphanol dissolved in the physiologically acceptable solvent-carrier, with the container having, and the contents therein being under, a breakable seal;
c. providing a disposable metered applicator of the intranasal drug delivery device, into which the container is inserted, the metered dispensing applicator having seal-breaking means for breaking the seal of the container, spraying means for forming a spray of the volume of liquid solution that is in the container, and delivery means for delivering at least 97% by volume and not more than 103% by volume of the predetermined dosage into a nasal cavity as a liquid spray, upon breaking of the seal of the container such that there is essentially no significant quantity of the therapeutic composition containing the pharmaceutically active agent remaining in the container or the dispensing applicator after application;
d. assembling the intranasal drug delivery device by inserting the container into the metered dispensing applicator;
e. inserting a contoured head portion of the metered applicator into a nasal cavity;
f. breaking the seal of the container with the seal-breaking means;
g. simultaneously withdrawing the liquid solution contents of the container and forming a liquid spray thereof with the spraying means; and
h. delivering the liquid spray through the delivery means to an outlet opening in the head portion of the metered dispensing applicator, which is in communication with the delivery means, from which the liquid spray, containing the unit-dosage of butorphanol, is released into the nasal cavity.

19. A pre-measured volume of a pharmaceutical drug dosage unit, having a predetermined amount of drug, for intranasal administration, as an atomized liquid spray, to a warm-blooded animal, of a pharmaceutically active agent that has been approved for use in producing a pharmacologically induced physiological response in the animal, the pharmaceutical dosage unit comprising:
a. at least one unit-dosage of the pharmaceutically active agent, selected from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, in liquid solution form, for delivery by intranasal administration as a liquid, each unit-dosage having a total volume and containing:
i. an effective amount of the pharmaceutically active agent for inducing the desired physiological response, so as to produce a desired level of bio-availability of the pharmaceutically active agent in the animal, after administration, the pharmaceutically active agent being present as a solute of a liquid solution; and
ii. a volume of a physiologically acceptable solvent-carrier for each unit-dosage of the pharmaceutically active agent solute, the solvent-carrier being selected on the basis of the solubility of the pharmaceutically active agent solute in the solvent-carrier;
such that the liquid solution of the pharmaceutically active agent in the physiologically acceptable solvent-carrier has a pre-determined concentration, whereby a pre-determined volume, not greater than from about 0.025 ml to about 0.75 ml, of the liquid solution, contains at least one unit-dosage of the effective amount of the pharmaceutically active agent; and
b. a liquid storage container, having at least one liquid storage compartment, for insertion into and use with a metered intranasal drug delivery device, for containing the volume of liquid solution of the at least one unit-dosage of the pharmaceutically active agent dissolved in the physiologically acceptable solvent-carrier, that provides at least one unit-dosage of the effective amount of the pharmaceutically active agent upon administration of the dosage, with the liquid storage container having, and the contents therein being under, a breakable seal.

20. A pharmaceutical drug dosage unit for providing a precisely measured dosage of a drug to a patient by intranasal administration thereto of the drug in the form of a liquid spray, the pharmaceutical drug dosage unit comprising:
a. at least one unit-dosage of a pharmaceutically active agent, selected from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, in liquid solution form, for delivery by intranasal administration to the patient, as an atomized liquid spray, the dosage unit having a volume which is a total volume of all unit-dosages contained in the dosage unit, such that each unit-dosage of the dosage unit contains:
i. an effective amount of the pharmaceutically active agent sufficient to induce a physiological response, the pharmaceutically active agent being present as a solute of a liquid solution; and
ii. a volume of a physiologically acceptable solvent-carrier for the pharmaceutically active agent solute, the solvent-carrier being selected on the basis of the solubility of the pharmaceutically active agent solute in the solvent-carrier;
such that the liquid solution of the pharmaceutically active agent in the physiologically acceptable solvent-carrier has a predetermined concentration, whereby a volume of the liquid solution, of from about 0.025 ml to about 0.75 ml, contains one unit-dosage of the effective amount of the pharmaceutically active agent; and
b. a container having at least one liquid storage compartment, for containing the volume of liquid solution of the at least one unit-dosage of the pharmaceutically active agent dissolved in the physiologically acceptable solvent-carrier, that delivers at least one unit-dosage of the effective amount of the pharmaceutically active agent upon administration of the dosage, as the contents of the compartment, with the container having, and the contents therein being under, a breakable seal.

21. A single-dose, single-use hydromorphone unit-dosage intranasal drug dosage unit comprising:
a. a single unit-dosage of hydromorphone, in liquid solution form, for delivery by intranasal administration to a person, as a liquid spray, each unit-dosage having a total volume not greater than about 2 ml, and containing:
i. an effective amount of hydromorphone, of from about 0.25 mg to about 10.0 mg, for inducing a systemic analgesic physiological response in the person, and to produce a desired level of bio-availability of the hydromorphone after administration, the hydromorphone being present as a solute of a liquid solution; and
ii. a volume of a physiologically acceptable solvent-carrier for the unit-dosage of the hydromorphone, the solvent-carrier being selected on the basis of the solubility of the hydromorphone therein;
such that the liquid solution of the hydromorphone in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the hydromorphone;
b. a disposable container, having at least one liquid storage compartment, for use in a metered applicator of an intranasal drug delivery device, for containing the volume of liquid solution of the one unit-dosage of the hydromorphone dissolved in the physiologically acceptable solvent-carrier, that provides the one unit-dosage of the effective amount of the hydromorphone upon intranasal administration of the dosage, with the container having, and the contents therein being under, a breakable seal, such that upon breaking of the seal, the contents of the container is released through the metered applicator into a nasal cavity as a liquid spray.

22. A single-dose, single-use butorphanol unit-dosage intranasal drug dosage delivery system comprising:
a. a single unit-dosage of butorphanol, in liquid solution form, for delivery by intranasal administration to a person, as a liquid spray, each unit-dosage having a total volume not greater than about 2 ml, and containing:
i. an effective amount of butorphanol, of from about 0.25 mg to about 10.0 mg, for inducing a systemic analgesic physiological response in the person, and to produce a desired level of bio-availability of the butorphanol after administration, the butorphanol being present as a solute of a liquid solution; and
ii. a volume of a physiologically acceptable solvent-carrier for the unit-dosage of the butorphanol, the solvent-carrier being selected on the basis of the solubility of the butorphanol therein;
such that the liquid solution of the butorphanol in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the butorphanol;
b. a disposable container, having at least one liquid storage compartment, for use in a metered applicator of an intranasal drug delivery device, for containing the volume of liquid solution of the one unit-dosage of the butorphanol dissolved in the physiologically acceptable solvent-carrier, with the container having, and the contents therein being under, a breakable seal, such that upon breaking of the seal, the contents of the container is released through the metered applicator into a nasal cavity as a liquid spray.

23. A method for preparing a pre-measured volume of a pharmaceutical drug dosage unit, containing a predetermined amount of a pharmaceutically active agent, for use in an intranasal drug delivery system, whereby the pharmaceutically active agent is intranasally administered, as a liquid spray, to a warm-blooded animal, for the purpose of producing a pharmacologically induced physiological response in the animal, the method comprising the steps of:
a. selecting a pharmaceutically active agent from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl and non-toxic pharmaceutically acceptable acid addition salts and metabolites thereof, that has been approved for use in producing the desired response, for use as a solute of a liquid solution;
b. determining an effective unit-dosage amount of the pharmaceutically active agent, for delivery by intranasal administration, so as to produce a desired level of bio-availability of the pharmaceutically active agent in the animal, after administration;
c. determining a physiologically acceptable solvent-carrier for the pharmaceutically active agent solute, the solvent-carrier being selected such that the pharmaceutically active agent is soluble therein, to the extent that a volume of liquid solution of the pharmaceutically active agent dissolved in the physiologically acceptable solvent-carrier, that contains the effective unit-dosage amount of the pharmaceutically active agent, is not greater than from about 0.025 ml to about 0.75 ml per unit-dosage;
d. forming a liquid solution of the pharmaceutically active agent in the physiologically acceptable solvent-carrier, with the liquid solution having a pre-determined concentration, such that a pre-determined volume of the liquid solution contains at least one unit-dosage of the effective amount of the pharmaceutically active agent;
e. placing a volume of liquid solution of the pharmaceutically active agent dissolved in the physiologically acceptable solvent-carrier, sufficient to provide at least one unit-dosage of the effective amount of the pharmaceutically active agent, into at least one liquid storage compartment of a liquid storage container for insertion into and use with a metered intranasal drug delivery device;
f. sealing the at least one liquid storage compartment of the liquid storage container; and
g. making the liquid storage container available for use together with a metered applicator of the intranasal drug delivery device for administration, as a liquid spray.

24. A method for the preparation of a pre-measured volume of a unit-drug-dosage amount of a pharmaceutically active agent, for administration as a liquid spray, to a warm-blooded animal, for the purpose of producing a pharmacologically induced physiological response in the animal, the method comprising the steps of:
a. preparing a unit-dosage of the pharmaceutically active agent, selected from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, in liquid solution form, for delivery by intranasal administration to the patient, as a liquid spray, the unit-dosage having a volume that contains:
i. an effective amount of the pharmaceutically active agent sufficient to induce a physiological response, the pharmaceutically active agent being present as a solute of a liquid solution; and
ii. a volume of a physiologically acceptable solvent-carrier for the pharmaceutically active agent solute, the solvent-carrier being selected on the basis of the solubility of the pharmaceutically active agent solute in the solvent-carrier;
such that the liquid solution of the pharmaceutically active agent in the physiologically acceptable solvent-carrier has a pre-determined concentration, whereby a volume of the liquid solution, of from about 0.025 ml to about 0.75 ml, contains one unit-dosage of the effective amount of the pharmaceutically active agent;
b. providing a container having at least one liquid storage compartment, for use with an applicator of a drug dosage delivery device, the compartment being for containing the volume of liquid solution of the unit-dosage of the pharmaceutically active agent dissolved in the physiologically acceptable solvent-carrier, as the contents of the compartment;
c. filling the compartment with the pre-determined volume of the liquid solution of the pharmaceutically active agent dissolved in the physiologically acceptable solvent-carrier; and
d. forming a breakable seal over the contents in the container.

25. A method for the preparation of a dosage-unit of hydromorphone for intranasal administration, as a liquid spray, to a person, utilizing a single unit-dosage sized container of hydromorphone, the container being disposable after use in an applicator of a drug delivery device, the method comprising the steps of:
a. providing a single unit-dosage of hydromorphone, in liquid solution form, for delivery by intranasal administration to a person, as an atomized liquid spray, the unit-dosage having a total volume not greater than about 2 ml, and containing:
i. an effective amount of hydromorphone, of from about 0.25 mg to about 10.0 mg, for inducing a systemic analgesic physiological response in the person, and to produce a desired level of bio-availability of the hydromorphone after administration, the hydromorphone being present as a solute of a liquid solution; and
ii. a volume of a physiologically acceptable solvent-carrier for the unit-dosage of the hydromorphone, the solvent-carrier being selected on the basis of the solubility of the hydromorphone therein;
such that the liquid solution of the hydromorphone in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the hydromorphone;
b. providing a disposable container having at least one liquid storage compartment, for use with an applicator of an intranasal drug delivery device, for containing the volume of liquid solution of the one unit-dosage of the hydromorphone dissolved in the physiologically acceptable solvent-carrier, that provides the one unit-dosage effective amount of the hydromorphone upon intranasal administration of the dosage, as the contents of the compartment;
c. filling the compartment with the pre-determined volume of the liquid solution of the hydromorphone dissolved in the physiologically acceptable solvent-carrier; and
d. forming a breakable seal over the contents in the container.

26. A method for the preparation of a dosage-unit of butorphanol, for intranasal administration, as a liquid spray, to a person, utilizing a single unit-dosage sized container of butorphanol, the container being disposable after use in an applicator of a drug delivery device, the method comprising the steps of:
a. providing a single unit-dosage of butorphanol, in liquid solution form, for delivery by intranasal administration to a person, as an atomized aerosol mist, the unit-dosage having a total volume not greater than about 2 ml, and containing:
i. an effective amount of butorphanol, of from about 0.25 mg to about 10.0 mg, for inducing a systemic analgesic physiological response in the person, and to produce a desired level of bio-availability of the butorphanol after administration, the butorphanol being present as a solute of a liquid solution; and
ii. a volume of a physiologically acceptable solvent-carrier for the unit-dosage of the butorphanol, the solvent-carrier being selected on the basis of the solubility of the butorphanol therein;
such that the liquid solution of the butorphanol in the physiologically acceptable solvent-carrier has a concentration of from about 8 mg/ml to about 12 mg/ml, whereby a volume, of from about 0.025 ml to about 0.75 ml, of the liquid solution, contains the unit-dosage of the effective amount of the butorphanol;
b. providing a disposable container having at least one liquid storage compartment, for use with an applicator of an intranasal drug delivery device, for containing the volume of liquid solution of the one unit-dosage of the butorphanol dissolved in the physiologically acceptable solvent-carrier, that provides the one unit-dosage effective amount of the butorphanol upon intranasal administration of the dosage, as the contents of the compartment;
c. filling the compartment with the pre-determined volume of the liquid solution of the butorphanol dissolved in the physiologically acceptable solvent-carrier; and
d. forming a breakable seal over the contents in the container.

27. A pharmaceutical drug dosage delivery system kit, for intranasal administration of a pharmaceutically active agent, as a liquid spray, to a warm-blooded animal, of a pre-measured volume of a unit drug dosage containing a predetermined amount of a pharmaceutically active agent that has been approved for use in producing a pharmacologically induced physiological response in the animal, the pharmaceutical drug dosage delivery system kit comprising:
a. at least one unit-dosage of a pharmaceutically active agent, selected from the group consisting of morphine, apomorphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol, pentazocine, naloxone, naltrexone, diprenorphine, nalmexone, cyprenorphine, alazocine, oxilorphan, cyclorphan, ketobemidone, apocodeine, profadol, cyclorphan, cyprenorphine, dihydromorphine, pholcodine, hydroxypethidine, fentanyl, sufentanil and alfentanyl, and non-toxic pharmaceutically acceptable acid addition salts and metabolites thereof, containing an effective amount of the pharmaceutically active agent sufficient to produce the desired pharmacologically induced physiological response in the animal, in a liquid solution together with an amount of a physiologically acceptable solvent-carrier in which the pharmaceutically active agent is soluble, such that a volume of the liquid solution containing the unit-dosage is not greater than about 2.0 ml, with the at least one unit-dosage being sealed in at least one liquid storage compartment of a disposable container from which the contents therein is releasable by breaking the seal of the container; and
b. at least one metered intranasal drug delivery device into which at least one container containing at least one unit-dosage of the pharmaceutically active agent in solution with a volume of the physiologically acceptable solvent-carrier is insertable, so that the seal of the container can be broken by the metered intranasal drug delivery device and the contents of the container thereby be released into a nasal cavity as an atomized liquid spray.

28. The pharmaceutical drug dosage delivery system kit according to claim 29, wherein there is a single, re-usable metered intranasal drug delivery device, and a plurality of containers.

29. The pharmaceutical drug dosage delivery system kit according to claim 30, wherein there are from 2 to 24 containers, each containing at least one unit-dosage of the pharmaceutically active agent, provided with the kit.

30. The pharmaceutical drug dosage delivery system kit according to claim 31, wherein the containers are packaged on a tray.

31. The pharmaceutical drug dosage delivery system kit according to claim 29, wherein there are from 1 to 24 each of containers and metered drug delivery devices.

32. The pharmaceutical drug dosage delivery system kit according to claim 29, wherein the metered drug delivery devices and containers are each pre-assembled with a container inserted in a metered drug delivery device, forming a plurality of ready-for-use units.

33. The pharmaceutical drug dosage delivery system kit according to claim 29, wherein the plurality of pre-assembled, ready-for-use units are packaged on a tray.

34. The pharmaceutical drug dosage delivery system kit according to claim 29, wherein the pharmaceutically active agent is hydromorphone.

35. The pharmaceutical drug dosage delivery system kit according to claim 29, wherein the pharmaceutically active agent is butorphanol.

36. A method for providing a pharmaceutical drug dosage delivery system kit, for intranasal administration of a pharmaceutically active agent, as a liquid spray, to a warm-blooded animal, of a pre-measured volume of a unit drug dosage containing a predetermined amount of a pharmaceutically active agent that has been approved for use in producing a pharmacologically induced physiological response in the animal, the method comprising:
a. providing at least one unit-dosage of a pharmaceutically active agent, selected from the group consisting of (*), and non-toxic pharmaceutically acceptable acid addition salts and metabolites thereof, containing an effective amount of the pharmaceutically active agent sufficient to produce the desired pharmacologically induced physiological response in the animal, in a liquid solution together with an amount of a physiologically acceptable solvent-carrier in which the pharmaceutically active agent is soluble, such that a volume of the liquid solution containing the unit-dosage is not greater than about 2.0 ml, with the at least one unit-dosage being sealed in a disposable container from which the contents therein is releasable by breaking the seal of the container; and
b. providing at least one metered intranasal drug delivery device into which at least one container containing at least one unit-dosage of the pharmaceutically active agent in solution with a volume of the physiologically acceptable solvent-carrier is insertable, so that the seal of the container can be broken by the metered intranasal drug delivery device and the contents of the container thereby be released into a nasal cavity as an atomized liquid spray.

37. A method of making a pharmaceutical drug dosage delivery system kit comprising making a plurality of pharmaceutical drug dosage delivery system units according to claim 1, and packaging them together.

38. The method according to claim 39, wherein there is a corresponding plurality of pharmaceutical unit-dosages and metered intranasal drug delivery applicator devices.

39. A method of making a pharmaceutical drug dosage delivery system kit comprising making a plurality of pharmaceutical dosage units according to the method of claim 25, and packaging them together with at least one intranasal drug delivery applicator device.

40. The method according to claim 41, wherein there is a corresponding plurality of intranasal drug dosage applicators to the plurality of pharmaceutical dosage units.
